(19) United States
(12) Patent Application Publication
(10) Pub. No.: US 2024/0071578 A1
(43) Pub. Date: Feb. 29, 2024

(54) UTILIZING DEEP REINFORCEMENT LEARNING FOR DISCOVERING NEW COMPOUNDS

(71) Applicant: Accenture Global Solutions Limited, Dublin (IE)

(72) Inventors: Rory McGRATH, Kildare Town (IE); Jeremiah HAYES, Dublin (IE); Xu ZHENG, Dublin (IE)

(21) Appl. No.: 17/821,916

(22) Filed: Aug. 24, 2022

(51) Int. Cl. G16C 20/70; G06N 3/08

(52) U.S. Cl. CPC G16C 20/70; G06N 3/08

(57) ABSTRACT

A device may receive source compound simplified molecular-input line-entry (SMILE) data, target compound SMILE data, and a latent space representing compounds, and may project the source compound SMILE data and the target compound SMILE data into the latent space to generate a source compound tensor and a target compound tensor, respectively. The device may process the source compound tensor, with one or more pretrained models, to determine a reward for the source compound tensor, and may determine, based on the reward, a direction and a magnitude to move in the latent space from the source compound tensor. The device may move the direction and the magnitude in the latent space to a new compound tensor, and may determine whether the new compound tensor matches the target compound tensor. The device may return a policy based on the new compound tensor matching the target compound tensor.

### BACKGROUND

Current methods of new drug discovery are time consuming and expensive. Machine learning may be utilized to discover new drugs. Machine learning is a type of artificial intelligence that allows software applications to become more accurate at predicting outcomes without being explicitly programmed.

### SUMMARY

Some implementations described herein relate to a method. The method may include receiving source compound simplified molecular-input line-entry (SMILE) data, target compound SMILE data, and a latent space representing compounds, and projecting the source compound SMILE data and the target compound SMILE data into the latent space to generate a source compound tensor and a target compound tensor, respectively. The method may include processing the source compound tensor, with one or more pretrained models, to determine a reward for the source compound tensor, and determining, based on the reward, a direction and a magnitude to move in the latent space from the source compound tensor. The method may include moving the direction and the magnitude in the latent space to a new compound tensor, and determining whether the new compound tensor matches the target compound tensor. The method may include returning a policy based on the new compound tensor matching the target compound tensor.

Some implementations described herein relate to a device. The device may include one or more memories and one or more processors coupled to the one or more memories. The one or more processors may be configured to receive source compound SMILE data, target compound SMILE data, and a latent space representing compounds, and project the source compound SMILE data and the target compound SMILE data into the latent space to generate a source compound tensor and a target compound tensor, respectively. The one or more processors may be configured to process the source compound tensor, with one or more pretrained models, to determine a reward for the source compound tensor, and determine, based on the reward, a direction and a magnitude to move in the latent space from the source compound tensor. The one or more processors may be configured to move the direction and the magnitude in the latent space to a new compound tensor, and determine whether the new compound tensor matches the target compound tensor. The one or more processors may be configured to return a policy based on the new compound tensor matching the target compound tensor or determine a new reward, a new direction, and a new magnitude for the new compound tensor based on the new compound tensor failing to match the target compound tensor.

Some implementations described herein relate to a non-transitory computer-readable medium that stores a set of instructions for a device. The set of instructions, when executed by one or more processors of the device, may cause the device to receive source compound SMILE data, target compound SMILE data, and a latent space representing compounds, and identify the source compound SMILE data. The set of instructions, when executed by one or more processors of the device, may cause the device to project the source compound SMILE data and the target compound SMILE data into the latent space to generate a source compound tensor and a target compound tensor, respectively, and process the source compound tensor, with one or more pretrained models, to determine a reward for the source compound tensor. The set of instructions, when executed by one or more processors of the device, may cause the device to determine, based on the reward, a direction and a magnitude to move in the latent space from the source compound tensor, and move the direction and the magnitude in the latent space to a new compound tensor. The set of instructions, when executed by one or more processors of the device, may cause the device to determine whether the new compound tensor matches the target compound tensor, and return a policy based on the new compound tensor matching the target compound tensor.

### BRIEF DESCRIPTION OF THE DRAWINGS

FIGS. 1A-1J are diagrams of an example implementation described herein.

FIG. 2 is a diagram of an example environment in which systems and/or methods described herein may be implemented.

FIG. 3 is a diagram of example components of one or more devices of FIG. 2.

FIG. 4 is a flowchart of an example process for utilizing deep reinforcement learning for discovering new compounds.

### DETAILED DESCRIPTION

The following detailed description of example implementations refers to the accompanying drawings. The same reference numbers in different drawings may identify the same or similar elements.

Machine learning may be utilized for early identification of drugs (e.g., compounds) with the greatest probability of being safe and effective, and for discerning and discarding potential compounds that are likely to fail at later stages of drug development. Current work in the field of drug discovery creates a latent space by training a variational autoencoder (VAE). However, the latent space generated by the VAE may include large dead areas that decode to invalid compounds (e.g., SMILE data). When interpolating between existing compounds in a latent space, the interpolation may not result in valid compounds. One technique for interpolating through the latent space is linear interpolation of compounds by following a shortest Euclidean path between latent representations. Another technique for interpolating through the latent space is spherical interpolation of compounds by following a circular arc lying on a surface of an n-dimensional sphere. In both techniques, the interpolation between two points may pass by areas with a low probability of generating a valid compound or a compound with desired properties.

Therefore, current techniques for utilizing machine learning to discover new drugs consume computing resources (e.g., processing resources, memory resources, communication resources, and/or the like), networking resources, and/or the like associated with improperly interpolating through a latent space, identifying invalid compounds based on improperly interpolating through the latent space, identifying compounds with undesired properties based on improperly interpolating through the latent space, performing useless research and development on invalid compounds, and/or the like.

Some implementations described herein relate to a policy system that utilizes deep reinforcement learning for discovering new compounds. For example, the policy system may receive source compound SMILE data, target compound SMILE data, and a latent space representing compounds, and may project the source compound SMILE data and the target compound SMILE data into the latent space to generate a source compound tensor and a target compound tensor, respectively. The policy system may process the source compound tensor, with one or more pretrained models, to determine a reward for the source compound tensor, and may determine, based on the reward, a direction and a magnitude to move in the latent space from the source compound tensor. The policy system may move the direction and the magnitude in the latent space to a new compound tensor, and may determine whether the new compound tensor matches the target compound tensor. The policy system may return a policy based on the new compound tensor matching the target compound tensor.

In this way, the policy system utilizes deep reinforcement learning for discovering new compounds. For example, the policy system may utilize deep reinforcement learning to interpolate through a latent space and discover valid compounds based on the interpolation. The policy system may utilize a deep reinforcement learning model to navigate the latent space and to generate valid compounds with desired properties. Thus, the policy system provide a much more targeted approach to compound discovery than the current techniques described above. This, in turn, conserves computing resources, networking resources, and/or the like that would otherwise have been consumed in improperly interpolating through a latent space, identifying invalid compounds based on improperly interpolating through the latent space, identifying compounds with undesired properties based on improperly interpolating through the latent space, performing useless research and development on invalid compounds, and/or the like.

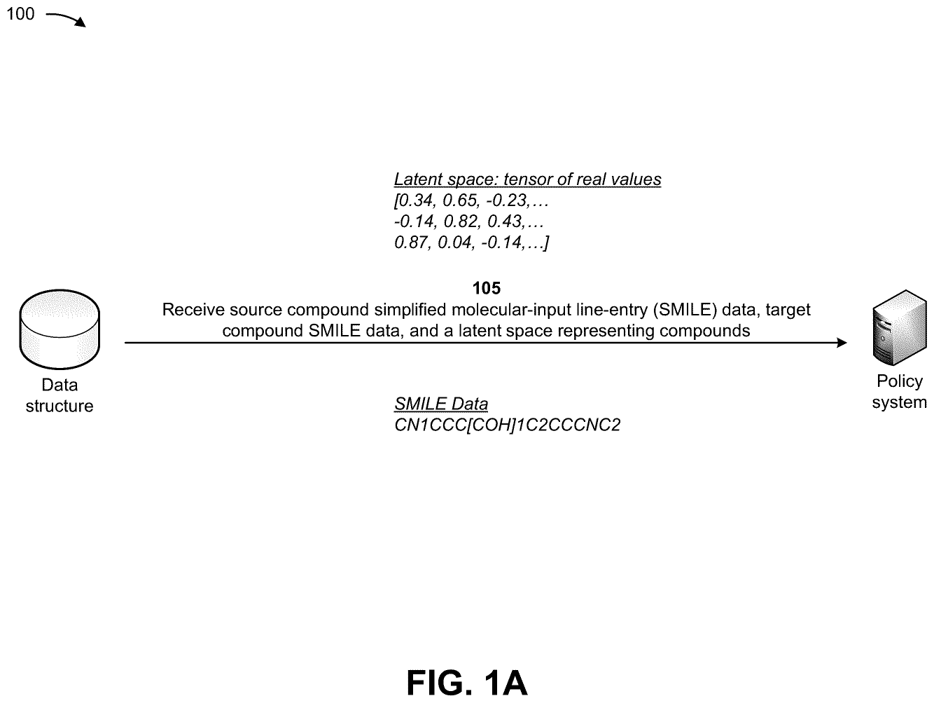

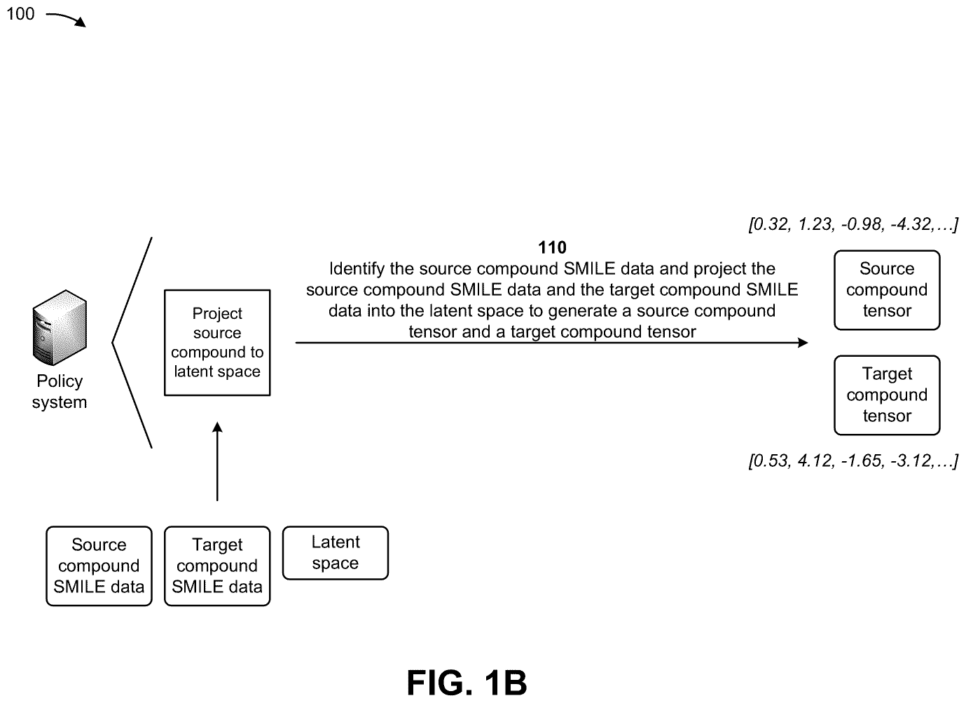

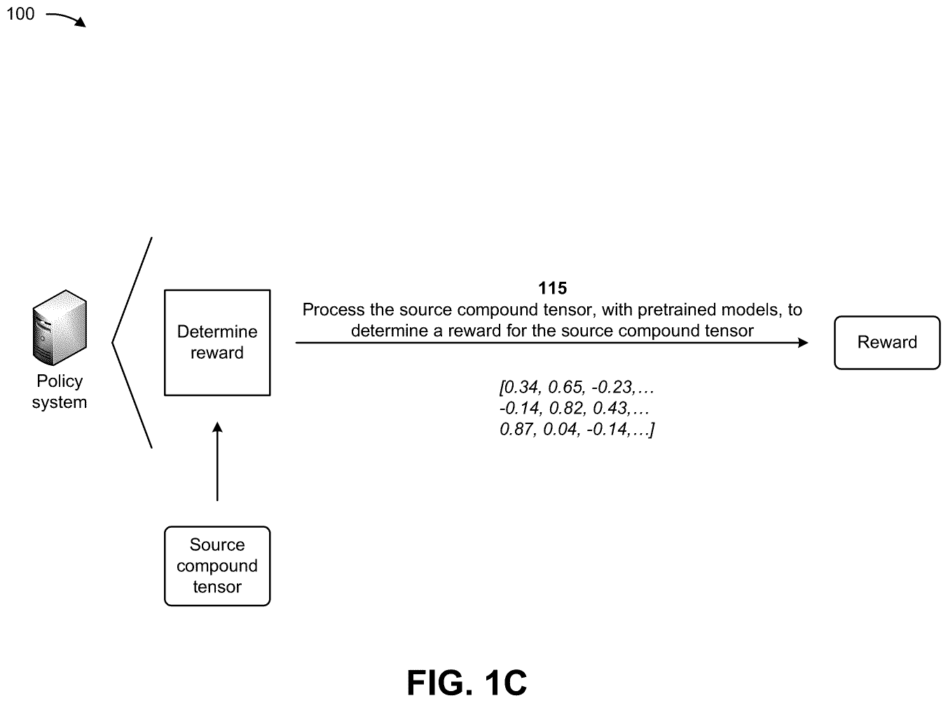

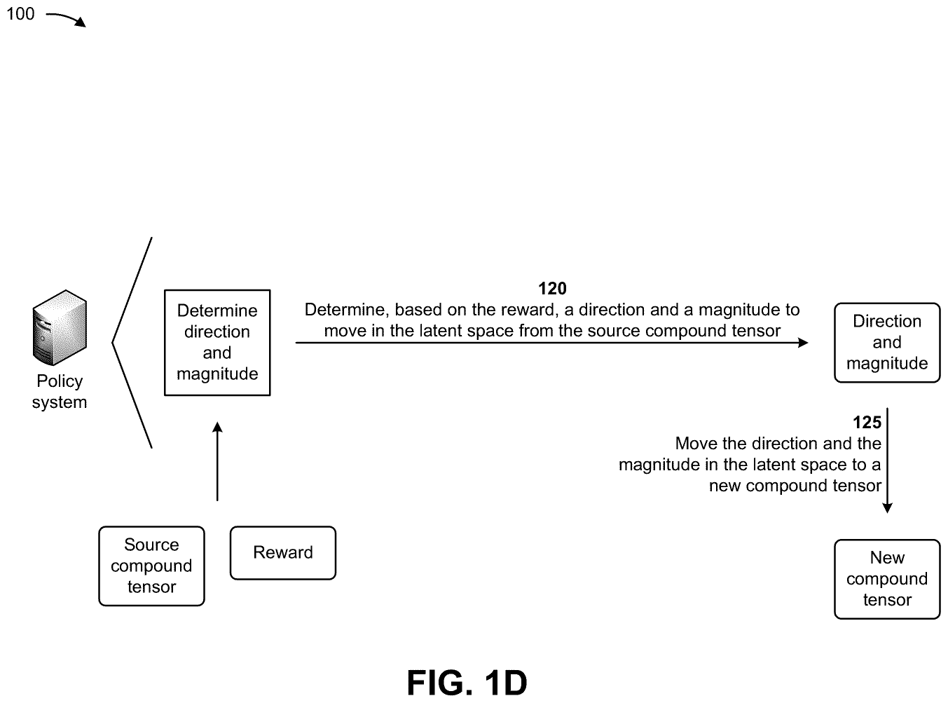

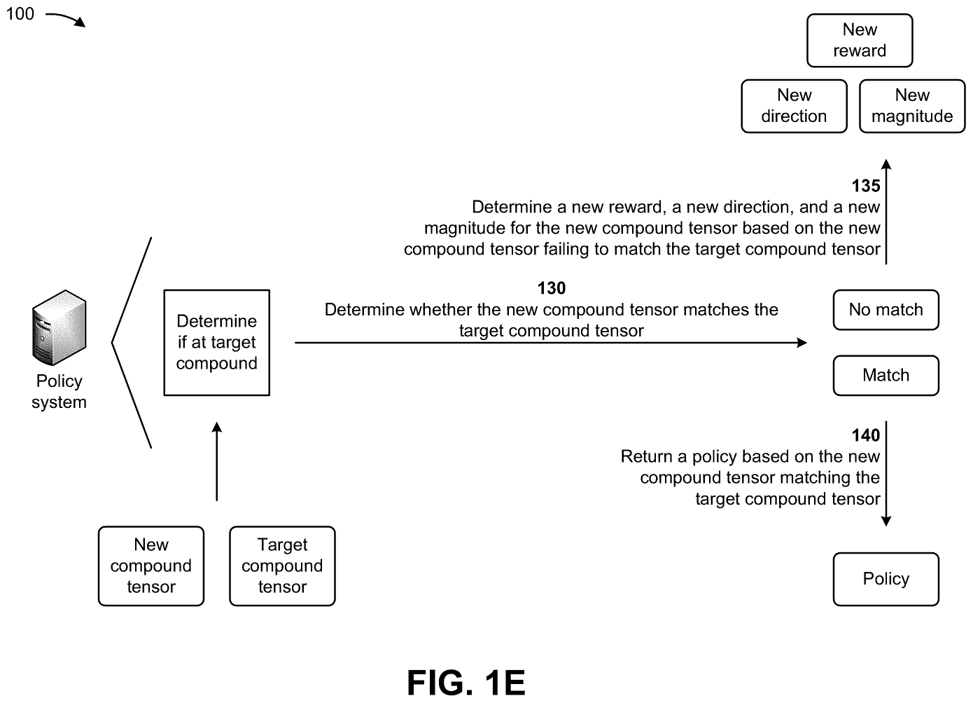

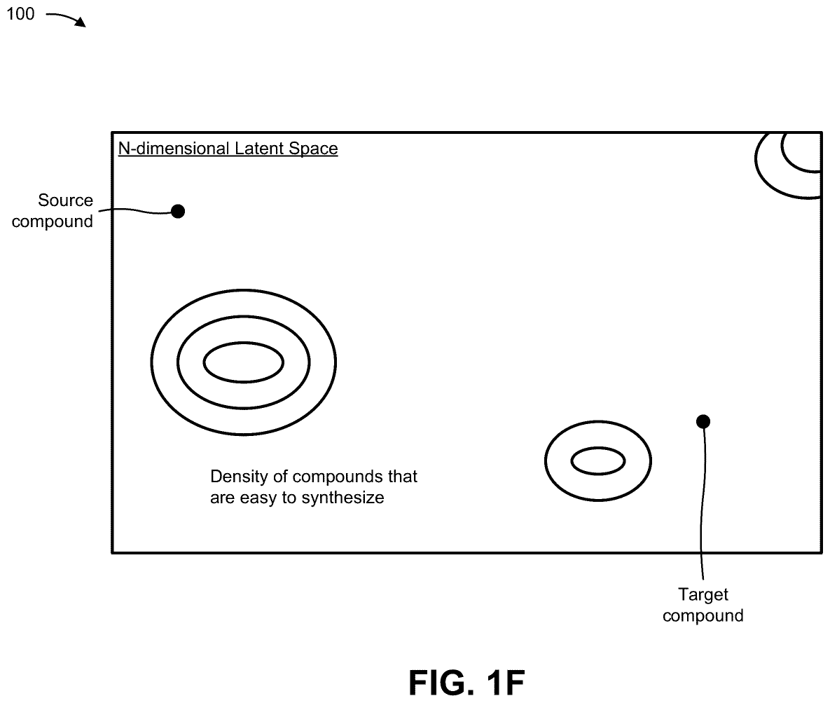

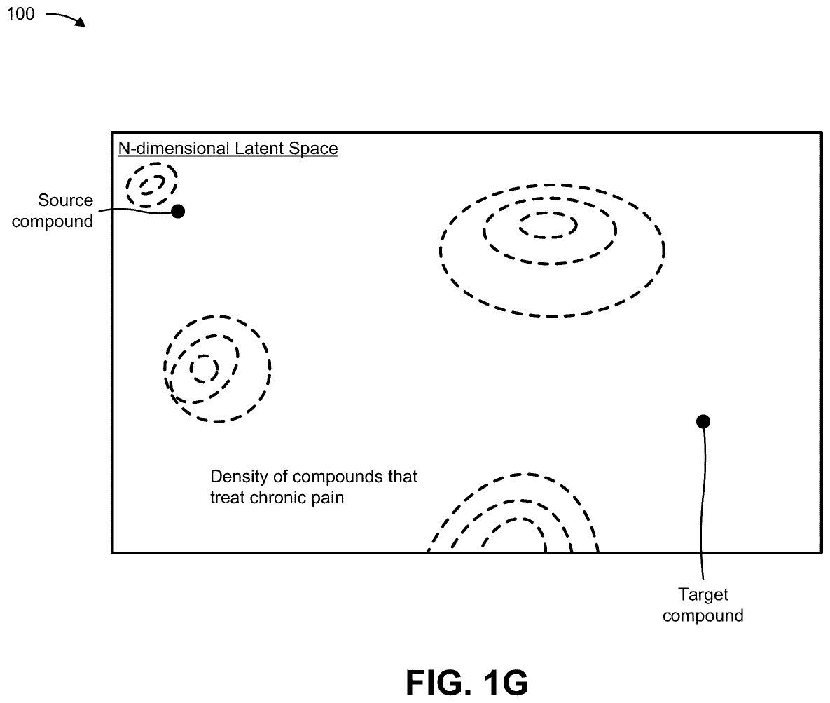

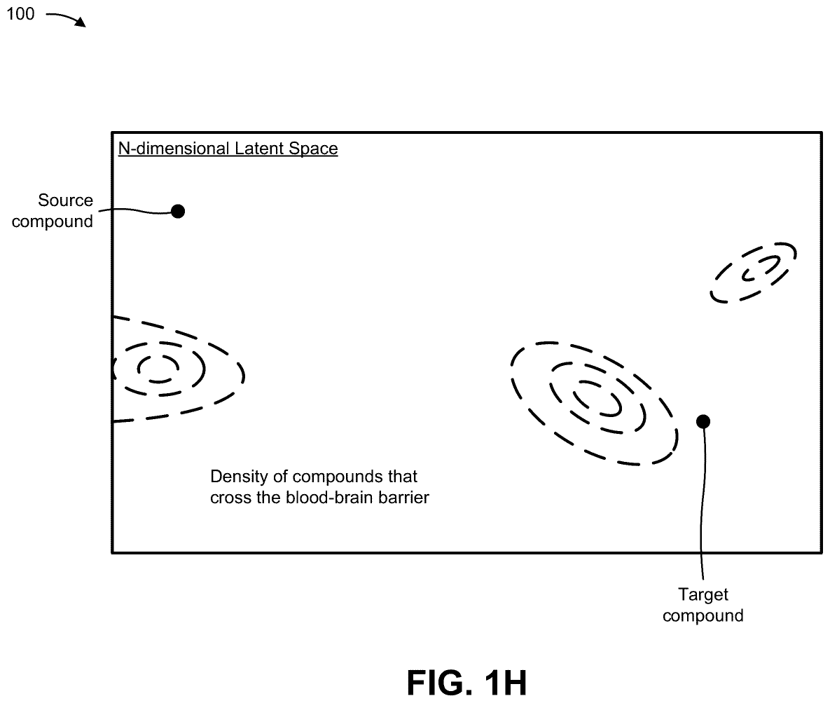

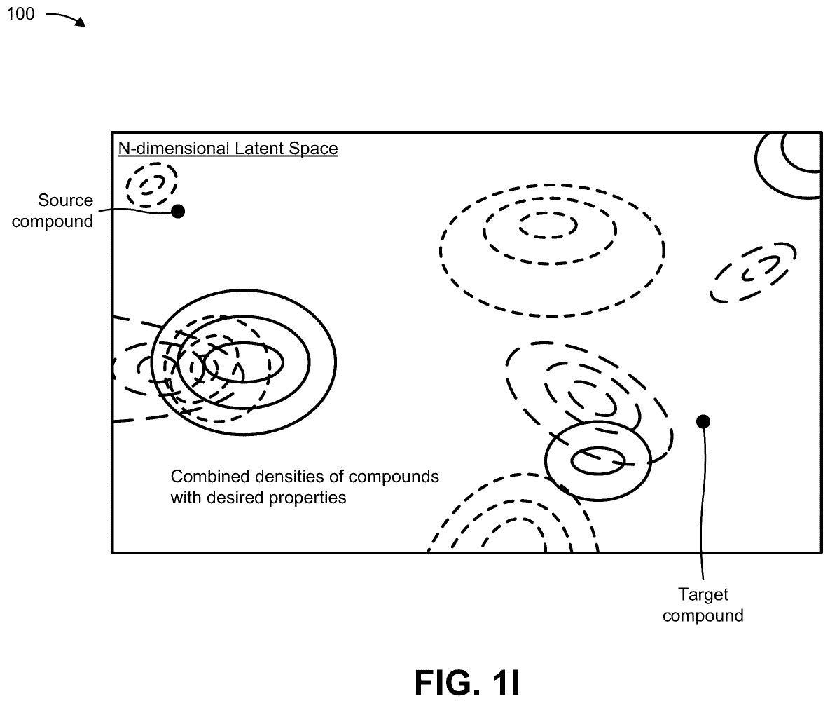

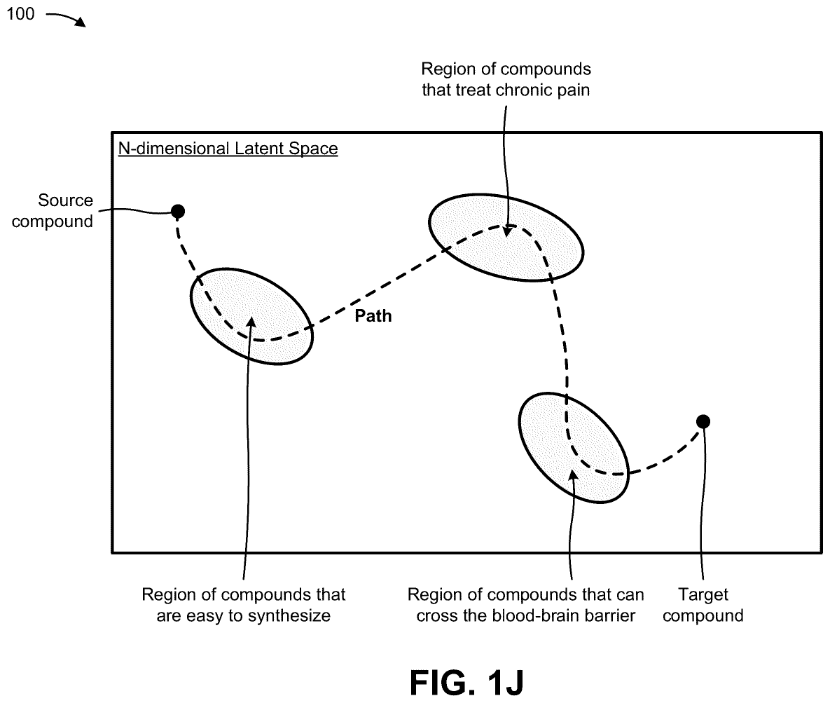

FIGS. 1A-1J are diagrams of an example 100 associated with utilizing deep reinforcement learning for discovering new compounds. As shown in FIGS. 1A-1J, example 100 includes a policy system associated with a data structure. The policy system may include a system that utilizes deep reinforcement learning for discovering new compounds. Further details of the policy system and the data structure are provided elsewhere herein.

As shown in FIG. 1A, and by reference number 105, the policy system may receive source compound SMILE data, target compound SMILE data, and a latent space representing compounds. For example, a data structure (e.g., a database, a table, a list, and/or the like) may store the source compound SMILE data, the target compound SMILE data, and the latent space. In some implementations, the policy system may continuously receive the source compound SMILE data, the target compound SMILE data, and/or the latent space from the data structure, may periodically receive the source compound SMILE data, the target compound SMILE data, and/or the latent space from the data structure, may receive the source compound SMILE data, the target compound SMILE data, and/or the latent space from the data structure based on providing a request to the data structure, and/or the like. The source compound SMILE data may include a specification in the form of a line notation for describing a structure of the source compound (e.g., OxyContin) using short ASCII strings (e.g., CNICCC[COH]1C2CCCNC2). The target compound SMILE data may include a specification in the form of a line notation for describing a structure of the target compound (e.g., Matulane) using short ASCII strings. The source compound SMILE data and the target compound SMILE data may include SMILE representations of compounds and diseases treated by the compounds or biological pathways of the compounds. The source compound SMILE data and the target compound SMILE data may be stored in an unstructured database, such as, for example, Stardog, Amazon Neptune, Neo4j, and/or the like. The source compound and the target compound may include known compounds with desirable properties. Although implementations are described in connection with SMILE data, the implementations may be utilized with any string-based representation of a compound, such as SMARTS, international chemical identifier (InChI), and/or the like.

A latent space is an abstract multi-dimensional space containing feature values that cannot be interpreted directly, but that encode a meaningful internal representation of externally observed events. The latent space may include a continuous high dimensional space into which the source compound SMILE data and the target compound SMILE data is projected. The latent space may accurately reconstruct the source compound SMILE data and the target compound SMILE data. In some implementations, the latent space may be pretrained with a model (e.g., a neural network model, a linear regression model, a decision tree model, a classification model, and/or the like) that predicts properties of SMILE data.

As shown in FIG. 1B, and by reference number 110, the policy system may identify the source compound SMILE data and may project the source compound SMILE data and the target compound SMILE data into the latent space to generate a source compound tensor and a target compound tensor, respectively. For example, the policy system may attempt to interpolate from the source compound SMILE data to the target compound SMILE data. Thus, the policy system may identify the source compound SMILE data as a starting location for the interpolation and may identify the target compound as a destination location for the interpolation. After identifying the starting location and the destination location, the policy system may project the source compound SMILE data and the target compound SMILE data into the trained latent space. The policy system may attempt to determine a policy, such as best route between the source compound SMILE data and the target compound SMILE data. Projecting the source compound SMILE data and the target compound SMILE data into the latent space may convert the string representations of the source compound SMILE data and the target compound SMILE data into multi-dimensional (e.g., n dimensional) tensors of real numbers (e.g., the source compound tensor and the target compound tensor, respectively).

As shown in FIG. 1C, and by reference number 115, the policy system may process the source compound tensor, with pretrained models, to determine a reward for the source compound tensor. For example, for each new point in the latent space a reward needs to be calculated by the policy system. Initially, the policy system may process the source compound tensor (e.g., a first new point in the latent space), with the pretrained models, to determine the reward for the source compound tensor. One advantage to using such an approach is that non-differentiable functions may be utilized by the policy system. The pretrained models may estimate desired property values (e.g., for regressions) or probabilities of having desired properties (e.g., for classifications) along with heuristics. For example, the pretrained models may estimate a variety of properties, such as an ability of a compound to treat chronic pain (E1), an ability of a compound to cross a blood-brain barrier (E2), and/or the like. The pretrained models may calculate a variety of heuristics, such as whether a compound decodes to a valid SMILE (H1), whether a compound has a log P of a water-octanal partition coefficient (H2), whether a compound is easy to synthesize (H3), whether a compound has a good drug likeliness score (H4), and/or the like. In some implementations, the policy may also utilize a distance from the target compound tensor (DJ) when calculating the reward for each new point (e.g., the source compound tensor initially).

The policy system may normalize the property estimates, the heuristic values, and the distance, and may linearly combine the normalized values together to calculate the reward for each new point. In some implementations, the policy system may assign positive operators to values to be maximized and negative operators to values to be minimized. For example, the policy system may calculate the reward for each new point as follows: Reward=E1+E2+H1+H2−H3+H4−D.

In some implementations, when processing the source compound tensor, with the one or more pretrained models, to determine the reward for the source compound tensor, the policy system may calculate one or more estimates (e.g., E1 and E2) associated with one or more properties of the source compound tensor, and may determine one or more heuristics (e.g., H1-H4) associated with the source compound tensor. The policy system may calculate a distance (e.g., D1) between the source compound tensor and the target compound tensor, and may determine the reward for the source compound tensor based on the one or more estimates, the one or more heuristics, and the distance. In some implementations, when determining the reward for the source compound tensor based on the one or more estimates, the one or more heuristics, and the distance, the policy system may combine the one or more estimates, the one or more heuristics, and the distance together to determine the reward for the source compound tensor.

As shown in FIG. 1D, and by reference number 120, the policy system may determine, based on the reward, a direction and a magnitude to move in the latent space from the source compound tensor. For example, once the reward is calculated, the policy may determine an action to perform, such determining the direction and the magnitude to move in the latent space from the source compound tensor. In some implementations, the policy system may move in any one of the multiple (e.g., n) dimensions of the latent space. For example, if the latent space has sixteen dimensions, the policy system may move in sixteen potential directions. With regard to the magnitude, the policy system may determine a distance (d) between the source compound tensor and the target compound tensor, and may divide the distance (d) by a value (k) to calculate one unit of movement (m). In one example, if k=100 and m=1, the policy system may determine the magnitude to be 1 m, 2 m, 3 m, and/or the like in any of then dimensions of the latent space, in a positive direction or a negative direction. This may result in n×3×2 possible movements (e.g., directions and magnitudes) according to the equation: dimensionality of the latent space×quantity of allowed movements×positive or negative directions.

In some implementations, when determining the direction and the magnitude to move in the latent space from the source compound tensor, the policy system may determine the direction based on a dimension of the latent space, and may determine the magnitude based on dimensions of the latent space, a quantity of allowed moves, and positive or negative directions.

As further shown in FIG. 1D, and by reference number 125, the policy system may move the direction and the magnitude in the latent space to a new compound tensor. For example, the policy system may move the direction and the magnitude, in the latent space, from the source compound tensor, and may arrive at new coordinates in the latent space. The policy system may identify the new coordinates as a new compound tensor.

As shown in FIG. 1E, and by reference number 130, the policy system may determine whether the new compound tensor matches the target compound tensor. For example, the policy system may determine whether the target compound tensor has been reached. In some implementations, when determining whether the target compound tensor has been reached, the policy system may determine whether the new compound tensor matches the target compound tensor. The policy system may determine that the target compound tensor has been reached when the new compound tensor matches the target compound tensor. Alternatively, the policy system may determine that the target compound has not been reached when the new compound tensor fails to match the target compound tensor.

As further shown in FIG. 1E, and by reference number 135, the policy system may determine a new reward, a new direction, and a new magnitude for the new compound tensor based on the new compound tensor failing to match the target compound tensor. For example, when the policy system determines that the new compound tensor fails to match the target compound tensor (e.g., that the target compound has not been reached), the policy system may repeat the functionality described above in connection with FIGS. 1C and 1D (e.g., reference numbers 115-125) for new compound tensors and until the target compound has been reached. For example, the policy system may process the new compound tensor, with the one or more pretrained models and based on the new compound tensor failing to match the target compound tensor, to determine another reward for the new compound tensor. The policy system may determine, based on the other reward, another direction and another magnitude to move in the latent space from the new compound tensor, and may move the other direction and the other magnitude in the latent space to another new compound tensor. The policy system may determine whether the other new compound tensor matches the target compound tensor. This process may continue until a new compound tensor matches the target compound tensor.

As further shown in FIG. 1E, and by reference number 140, the policy system may return a policy based on the new compound tensor matching the target compound tensor. For example, when the policy system determines that the new compound tensor matches the target compound tensor (e.g., that the target compound has been reached), the policy system may return (e.g., output) the policy. In some implementations, the policy includes a route between the source compound tensor and the target compound tensor that identifies one or more compounds that satisfy one or more properties. An example of such a route is described below in connection with FIG. 1J. The policy system may determine the policy based on executing multiple simulations that traverse the multi-dimensional latent space from the source compound tensor to the target compound tensor while satisfying desired properties (e.g., easy to synthesize, treat chronic pain, cross the blood-brain barrier, and/or the like).

In some implementations, the policy system may perform one or more actions based on the policy. For example, the policy system may identify one or more new compounds based on the policy, and may provide data identifying the one or more new compounds for display to a user (e.g., researcher) of the policy system. In another example, the policy system may identify one or more new compound tensors based on the policy, and may generate new compound SMILE data based on the one or more new compound tensors. The policy system may store the new compound SMILE data in the data structure, may provide the new compound SMILE data for display to the user of the policy system, and/or the like. In this way, the policy system may aid the user in new drug discovery associated with the source compound and the target compound.

FIG. 1F depicts the source compound tensor and the target compound tensor in the latent space (e.g., a trained n-dimensional latent space). FIG. 1F also depicts a density of compounds (e.g., in the latent space) that are easy to synthesize, which corresponds to a heuristic (e.g., whether a compound is easy to synthesize (H3)) calculated by the pretrained models of the policy system.

FIG. 1G depicts the source compound tensor and the target compound tensor in the latent space. FIG. 1G also depicts a density of compounds (e.g., in the latent space) that treat chronic pain, which corresponds to an estimate (e.g., an ability of a compound to treat chronic pain (E1)) calculated by the pretrained models of the policy system.

FIG. 1H depicts the source compound tensor and the target compound tensor in the latent space. FIG. 1H also depicts a density of compounds (e.g., in the latent space) that cross the blood-brain barrier, which corresponds to an estimate (e.g., an ability of a compound to cross a blood-brain barrier (E2)) calculated by the pretrained models of the policy system.

FIG. 1I depicts the source compound tensor and the target compound tensor in the latent space. FIG. 1I also depicts a combined densities of compounds (e.g., in the latent space) with the desired properties (e.g., easy to synthesize, treat chronic pain, and cross the blood-brain barrier).

FIG. 1J depicts the source compound tensor and the target compound tensor in the latent space. FIG. 1J also depicts, in the latent space, a region of compounds that are easy to synthesize, a region of compounds that treat chronic pain, and a region of compounds that can cross the blood-brain barrier. As further shown, a path may be interpolated between the source compound tensor and the target compound tensor. The path may pass through the region of compounds that are easy to synthesize, the region of compounds that treat chronic pain, and the region of compounds that can cross the blood-brain barrier. Thus, the path may include valid compounds associated with the source compound and the target compound.

In this way, the policy system utilizes deep reinforcement learning for discovering new compounds. For example, the policy system may utilize deep reinforcement learning to interpolate through a latent space and discover valid compounds based on the interpolation. The policy system may utilize a deep reinforcement learning model to navigate the latent space and to generate valid compounds with desired properties. Thus, the policy system provides a much more targeted approach to compound discovery than the current techniques described above. This, in turn, conserves computing resources, networking resources, and/or the like that would otherwise have been consumed in improperly interpolating through a latent space, identifying invalid compounds based on improperly interpolating through the latent space, identifying compounds with undesired properties based on improperly interpolating through the latent space, performing useless research and development on invalid compounds, and/or the like.

As indicated above, FIGS. 1A-1J are provided as an example. Other examples may differ from what is described with regard to FIGS. 1A-1J. The number and arrangement of devices shown in FIGS. 1A-1J are provided as an example. In practice, there may be additional devices, fewer devices, different devices, or differently arranged devices than those shown in FIGS. 1A-1J. Furthermore, two or more devices shown in FIGS. 1A-1J may be implemented within a single device, or a single device shown in FIGS. 1A-1J may be implemented as multiple, distributed devices. Additionally, or alternatively, a set of devices (e.g., one or more devices) shown in FIGS. 1A-1J may perform one or more functions described as being performed by another set of devices shown in FIGS. 1A-1J.

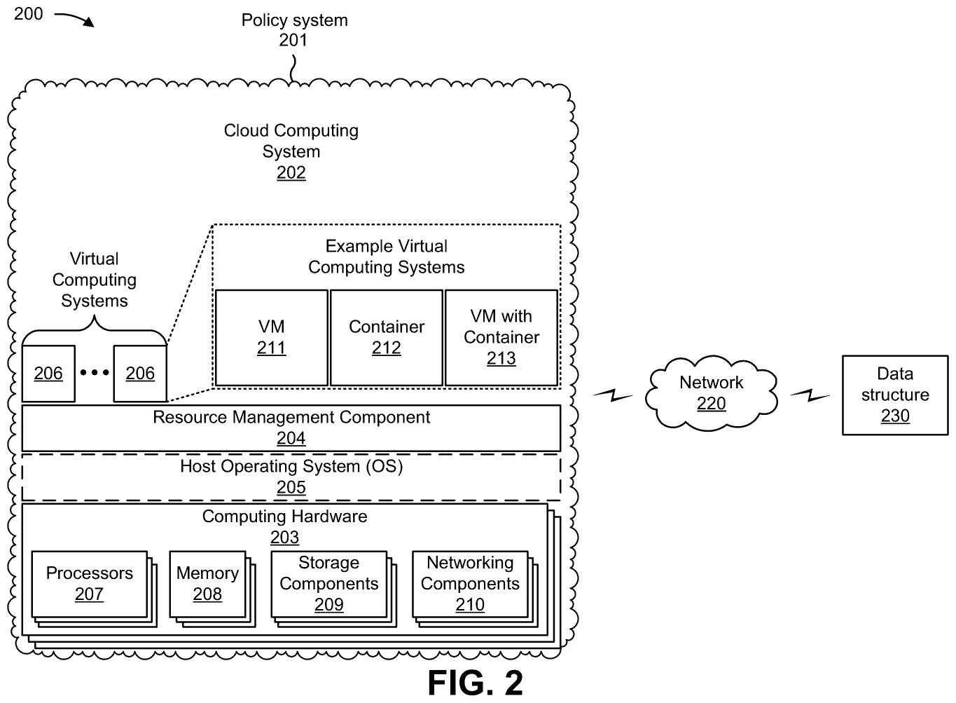

FIG. 2 is a diagram of an example environment 200 in which systems and/or methods described herein may be implemented. As shown in FIG. 2, the environment 200 may include a policy system 201, which may include one or more elements of and/or may execute within a cloud computing system 202. The cloud computing system 202 may include one or more elements 203-213, as described in more detail below. As further shown in FIG. 2, the environment 200 may include a network 220 and/or a data structure 230. Devices and/or elements of the environment 200 may interconnect via wired connections and/or wireless connections.

The cloud computing system 202 includes computing hardware 203, a resource management component 204, a host operating system (OS) 205, and/or one or more virtual computing systems 206. The resource management component 204 may perform virtualization (e.g., abstraction) of the computing hardware 203 to create the one or more virtual computing systems 206. Using virtualization, the resource management component 204 enables a single computing device (e.g., a computer, a server, and/or the like) to operate like multiple computing devices, such as by creating multiple isolated virtual computing systems 206 from the computing hardware 203 of the single computing device. In this way, the computing hardware 203 can operate more efficiently, with lower power consumption, higher reliability, higher availability, higher utilization, greater flexibility, and lower cost than using separate computing devices.

The computing hardware 203 includes hardware and corresponding resources from one or more computing devices. For example, the computing hardware 203 may include hardware from a single computing device (e.g., a single server) or from multiple computing devices (e.g., multiple servers), such as multiple computing devices in one or more data centers. As shown, the computing hardware 203 may include one or more processors 207, one or more memories 208, one or more storage components 209, and/or one or more networking components 210. Examples of a processor, a memory, a storage component, and a networking component (e.g., a communication component) are described elsewhere herein.

The resource management component 204 includes a virtualization application (e.g., executing on hardware, such as the computing hardware 203) capable of virtualizing the computing hardware 203 to start, stop, and/or manage the one or more virtual computing systems 206. For example, the resource management component 204 may include a hypervisor (e.g., a bare-metal or Type 1 hypervisor, a hosted or Type 2 hypervisor, and/or the like) or a virtual machine monitor, such as when the virtual computing systems 206 are virtual machines 211. Additionally, or alternatively, the resource management component 204 may include a container manager, such as when the virtual computing systems 206 are containers 212. In some implementations, the resource management component 204 executes within and/or in coordination with a host operating system 205.

A virtual computing system 206 includes a virtual environment that enables cloud-based execution of operations and/or processes described herein using computing hardware 203. As shown, a virtual computing system 206 may include a virtual machine 211, a container 212, a hybrid environment 213 that includes a virtual machine and a container, and/or the like. A virtual computing system 206 may execute one or more applications using a file system that includes binary files, software libraries, and/or other resources required to execute applications on a guest operating system (e.g., within the virtual computing system 206) or the host operating system 205.

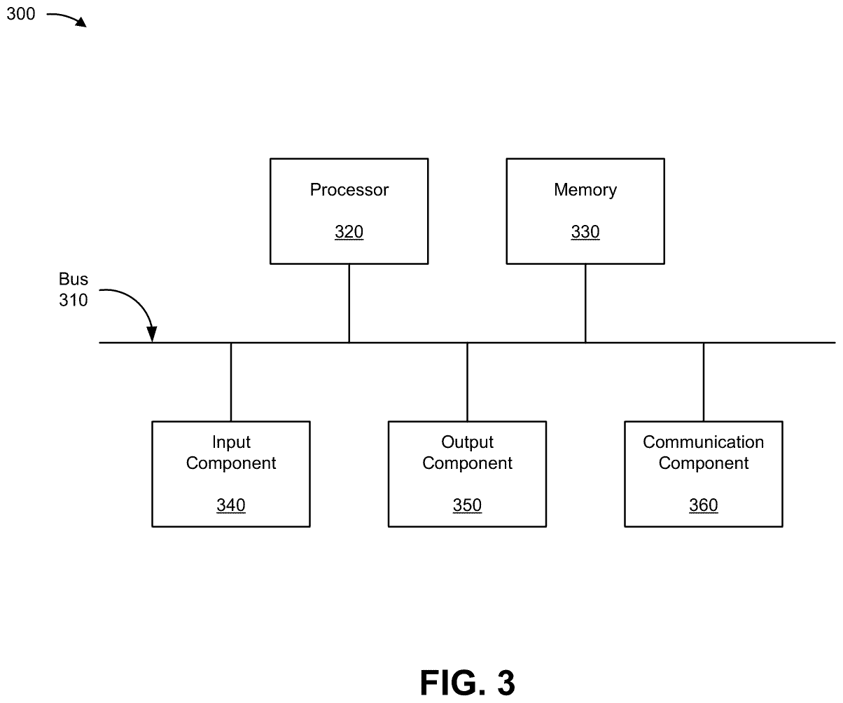

Although the policy system 201 may include one or more elements 203-213 of the cloud computing system 202, may execute within the cloud computing system 202, and/or may be hosted within the cloud computing system 202, in some implementations, the policy system 201 may not be cloud-based (e.g., may be implemented outside of a cloud computing system) or may be partially cloud-based. For example, the policy system 201 may include one or more devices that are not part of the cloud computing system 202, such as device 300 of FIG. 3, which may include a standalone server or another type of computing device. The policy system 201 may perform one or more operations and/or processes described in more detail elsewhere herein.

The network 220 includes one or more wired and/or wireless networks. For example, the network 220 may include a cellular network, a public land mobile network (PLMN), a local area network (LAN), a wide area network (WAN), a private network, the Internet, and/or the like, and/or a combination of these or other types of networks. The network 220 enables communication among the devices of the environment 200.

The data structure 230 may include one or more devices capable of receiving, generating, storing, processing, and/or providing information, as described elsewhere herein. The data structure 230 may include a communication device and/or a computing device. For example, the data structure 230 may include a database, a server, a database server, an application server, a client server, a web server, a host server, a proxy server, a virtual server (e.g., executing on computing hardware), a server in a cloud computing system, a device that includes computing hardware used in a cloud computing environment, or a similar type of device. The data structure 230 may communicate with one or more other devices of the environment 200, as described elsewhere herein.

The number and arrangement of devices and networks shown in FIG. 2 are provided as an example. In practice, there may be additional devices and/or networks, fewer devices and/or networks, different devices and/or networks, or differently arranged devices and/or networks than those shown in FIG. 2. Furthermore, two or more devices shown in FIG. 2 may be implemented within a single device, or a single device shown in FIG. 2 may be implemented as multiple, distributed devices. Additionally, or alternatively, a set of devices (e.g., one or more devices) of the environment 200 may perform one or more functions described as being performed by another set of devices of the environment 200.

FIG. 3 is a diagram of example components of a device 300, which may correspond to the policy system 201 and/or the data structure 230. In some implementations, the policy system 201 and/or the data structure 230 may include one or more devices 300 and/or one or more components of the device 300. As shown in FIG. 3, the device 300 may include a bus 310, a processor 320, a memory 330, an input component 340, an output component 350, and a communication component 360.

The bus 310 includes a component that enables wired and/or wireless communication among the components of device 300. The processor 320 includes a central processing unit, a graphics processing unit, a microprocessor, a controller, a microcontroller, a digital signal processor, a field-programmable gate array, an application-specific integrated circuit, and/or another type of processing component. The processor 320 is implemented in hardware, firmware, or a combination of hardware and software. In some implementations, the processor 320 includes one or more processors capable of being programmed to perform a function. The memory 330 includes a random-access memory, a read only memory, and/or another type of memory (e.g., a flash memory, a magnetic memory, and/or an optical memory).

The input component 340 enables the device 300 to receive input, such as user input and/or sensed inputs. For example, the input component 340 may include a touch screen, a keyboard, a keypad, a mouse, a button, a microphone, a switch, a sensor, a global positioning system component, an accelerometer, a gyroscope, an actuator, and/or the like. The output component 350 enables the device 300 to provide output, such as via a display, a speaker, and/or one or more light-emitting diodes. The communication component 360 enables the device 300 to communicate with other devices, such as via a wired connection and/or a wireless connection. For example, the communication component 360 may include a receiver, a transmitter, a transceiver, a modem, a network interface card, an antenna, and/or the like.

The device 300 may perform one or more processes described herein. For example, a non-transitory computer-readable medium (e.g., the memory 330) may store a set of instructions (e.g., one or more instructions, code, software code, program code, and/or the like) for execution by the processor 320. The processor 320 may execute the set of instructions to perform one or more processes described herein. In some implementations, execution of the set of instructions, by one or more processors 320, causes the one or more processors 320 and/or the device 300 to perform one or more processes described herein. In some implementations, hardwired circuitry may be used instead of or in combination with the instructions to perform one or more processes described herein. Thus, implementations described herein are not limited to any specific combination of hardware circuitry and software.

The number and arrangement of components shown in FIG. 3 are provided as an example. The device 300 may include additional components, fewer components, different components, or differently arranged components than those shown in FIG. 3. Additionally, or alternatively, a set of components (e.g., one or more components) of the device 300 may perform one or more functions described as being performed by another set of components of the device 300.

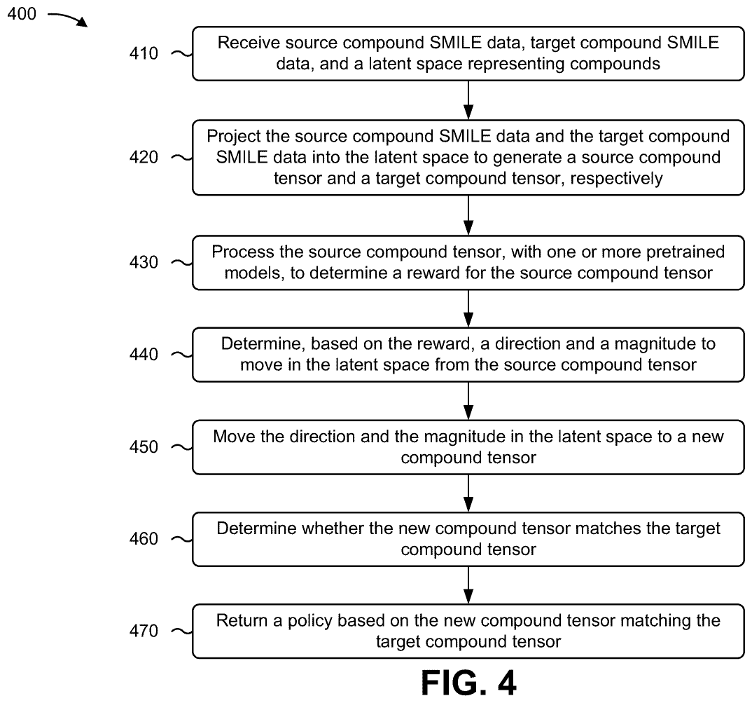

FIG. 4 is a flowchart of an example process 400 for utilizing deep reinforcement learning for discovering new compounds. In some implementations, one or more process blocks of FIG. 4 may be performed by a device (e.g., the policy system 201). In some implementations, one or more process blocks of FIG. 4 may be performed by another device or a group of devices separate from or including the device. Additionally, or alternatively, one or more process blocks of FIG. 4 may be performed by one or more components of the device 300, such as the processor 320, the memory 330, the input component 340, the output component 350, and/or the communication component 360.

As shown in FIG. 4, process 400 may include receiving source compound SMILE data, target compound SMILE data, and a latent space representing compounds (block 410). For example, the device may receive source compound SMILE data, target compound SMILE data, and a latent space representing compounds, as described above. In some implementations, the latent space is pretrained with a model that predicts properties of SMILE data.

As further shown in FIG. 4, process 400 may include projecting the source compound SMILE data and the target compound SMILE data into the latent space to generate a source compound tensor and a target compound tensor, respectively (block 420). For example, the device may project the source compound SMILE data and the target compound SMILE data into the latent space to generate a source compound tensor and a target compound tensor, respectively, as described above. In some implementations, each of the source compound tensor and the target compound tensor is a multi-dimensional tensor of real numbers.

As further shown in FIG. 4, process 400 may include processing the source compound tensor, with one or more pretrained models, to determine a reward for the source compound tensor (block 430). For example, the device may process the source compound tensor, with one or more pretrained models, to determine a reward for the source compound tensor, as described above. In some implementations, processing the source compound tensor, with the one or more pretrained models, to determine the reward for the source compound tensor includes calculating one or more estimates associated with one or more properties of the source compound tensor, determining one or more heuristics associated with the source compound tensor, calculating a distance between the source compound tensor and the target compound tensor, and determining the reward for the source compound tensor based on the one or more estimates, the one or more heuristics, and the distance. In some implementations, determining the reward for the source compound tensor based on the one or more estimates, the one or more heuristics, and the distance includes combining the one or more estimates, the one or more heuristics, and the distance together to determine the reward for the source compound tensor.

In some implementations, each of the one or more pretrained models is a deep reinforcement learning model.

As further shown in FIG. 4, process 400 may include determining, based on the reward, a direction and a magnitude to move in the latent space from the source compound tensor (block 440). For example, the device may determine, based on the reward, a direction and a magnitude to move in the latent space from the source compound tensor, as described above. In some implementations, determining the direction and the magnitude to move in the latent space from the source compound tensor includes determining the direction based on a dimension of the latent space, and determining the magnitude based on dimensions of the latent space, a quantity of allowed moves, and positive or negative directions.

As further shown in FIG. 4, process 400 may include moving the direction and the magnitude in the latent space to a new compound tensor (block 450). For example, the device may move the direction and the magnitude in the latent space to a new compound tensor, as described above.

As further shown in FIG. 4, process 400 may include determining whether the new compound tensor matches the target compound tensor (block 460). For example, the device may determine whether the new compound tensor matches the target compound tensor, as described above.

As further shown in FIG. 4, process 400 may include returning a policy based on the new compound tensor matching the target compound tensor (block 470). For example, the device may return a policy based on the new compound tensor matching the target compound tensor, as described above. In some implementations, the policy includes a route between the source compound tensor and the target compound tensor that identifies one or more compounds that satisfy one or more properties.

In some implementations, process 400 includes identifying the source compound SMILE data prior to projecting the source compound SMILE data into the latent space. In some implementations, process 400 includes determining a new reward, a new direction, and a new magnitude for the new compound tensor based on the new compound tensor failing to match the target compound tensor.

In some implementations, process 400 includes processing the new compound tensor, with the one or more pretrained models and based on the new compound tensor failing to match the target compound tensor, to determine another reward for the new compound tensor; determining, based on the other reward, another direction and another magnitude to move in the latent space from the new compound tensor; moving the other direction and the other magnitude in the latent space to another new compound tensor; determining whether the other new compound tensor matches the target compound tensor; and returning another policy based on the other new compound tensor matching the target compound tensor.

In some implementations, process 400 includes identifying one or more new compounds based on the policy, and providing data identifying the one or more new compounds for display. In some implementations, process 400 includes identifying one or more new compound tensors based on the policy, generating one or more new compound SMILE data based on the one or more new compound tensors, and providing the one or more new compound SMILE data for display.

Although FIG. 4 shows example blocks of process 400, in some implementations, process 400 may include additional blocks, fewer blocks, different blocks, or differently arranged blocks than those depicted in FIG. 4. Additionally, or alternatively, two or more of the blocks of process 400 may be performed in parallel.

The foregoing disclosure provides illustration and description but is not intended to be exhaustive or to limit the implementations to the precise form disclosed. Modifications may be made in light of the above disclosure or may be acquired from practice of the implementations.

As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code—it being understood that software and hardware can be used to implement the systems and/or methods based on the description herein.

As used herein, satisfying a threshold may, depending on the context, refer to a value being greater than the threshold, greater than or equal to the threshold, less than the threshold, less than or equal to the threshold, equal to the threshold, and/or the like, depending on the context.

Although particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

In the preceding specification, various example embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A method, comprising:
receiving, by a device, source compound simplified molecular-input line-entry (SMILE) data, target compound SMILE data, and a latent space representing compounds;
projecting, by the device, the source compound SMILE data and the target compound SMILE data into the latent space to generate a source compound tensor and a target compound tensor, respectively;
processing, by the device, the source compound tensor, with one or more pretrained models, to determine a reward for the source compound tensor;
determining, by the device and based on the reward, a direction and a magnitude to move in the latent space from the source compound tensor;
moving, by the device, the direction and the magnitude in the latent space to a new compound tensor;
determining, by the device, whether the new compound tensor matches the target compound tensor; and
returning, by the device, a policy based on the new compound tensor matching the target compound tensor.

2. The method of claim 1, further comprising:
identifying the source compound SMILE data prior to projecting the source compound SMILE data into the latent space.

3. The method of claim 1, further comprising:
determining a new reward, a new direction, and a new magnitude for the new compound tensor based on the new compound tensor failing to match the target compound tensor.

4. The method of claim 1, wherein each of the source compound tensor and the target compound tensor is a multi-dimensional tensor of real numbers.

5. The method of claim 1, wherein processing the source compound tensor, with the one or more pretrained models, to determine the reward for the source compound tensor comprises:
calculating one or more estimates associated with one or more properties of the source compound tensor;
determining one or more heuristics associated with the source compound tensor;
calculating a distance between the source compound tensor and the target compound tensor; and
determining the reward for the source compound tensor based on the one or more estimates, the one or more heuristics, and the distance.

6. The method of claim 5, wherein determining the reward for the source compound tensor based on the one or more estimates, the one or more heuristics, and the distance comprises:
combining the one or more estimates, the one or more heuristics, and the distance together to determine the reward for the source compound tensor.

7. The method of claim 1, wherein determining the direction and the magnitude to move in the latent space from the source compound tensor comprises:
determining the direction based on a dimension of the latent space; and
determining the magnitude based on dimensions of the latent space, a quantity of allowed moves, and positive or negative directions.

8. A device, comprising:
one or more memories; and
one or more processors, coupled to the one or more memories, configured to:
receive source compound simplified molecular-input line-entry (SMILE) data, target compound SMILE data, and a latent space representing compounds;
project the source compound SMILE data and the target compound SMILE data into the latent space to generate a source compound tensor and a target compound tensor, respectively;
process the source compound tensor, with one or more pretrained models, to determine a reward for the source compound tensor;
determine, based on the reward, a direction and a magnitude to move in the latent space from the source compound tensor;
move the direction and the magnitude in the latent space to a new compound tensor;
determine whether the new compound tensor matches the target compound tensor; and
return a policy based on the new compound tensor matching the target compound tensor or determine a new reward, a new direction, and a new magnitude for the new compound tensor based on the new compound tensor failing to match the target compound tensor.

9. The device of claim 8, wherein the one or more processors are further configured to:
process the new compound tensor, with the one or more pretrained models and based on the new compound tensor failing to match the target compound tensor, to determine another reward for the new compound tensor;
determine, based on the other reward, another direction and another magnitude to move in the latent space from the new compound tensor;
move the other direction and the other magnitude in the latent space to another new compound tensor;
determine whether the other new compound tensor matches the target compound tensor; and
return another policy based on the other new compound tensor matching the target compound tensor.

10. The device of claim 8, wherein each of the one or more pretrained models is a deep reinforcement learning model.

11. The device of claim 8, wherein the policy includes a route between the source compound tensor and the target compound tensor that identifies one or more compounds that satisfy one or more properties.

12. The device of claim 8, wherein the one or more processors are further configured to:
identify one or more new compounds based on the policy; and
provide data identifying the one or more new compounds for display.

13. The device of claim 8, wherein the one or more processors are further configured to:
identify one or more new compound tensors based on the policy;
generate one or more new compound SMILE data based on the one or more new compound tensors; and
provide the one or more new compound SMILE data for display.

14. The device of claim 8, wherein the latent space is pretrained with a model that predicts properties of SMILE data.

15. A non-transitory computer-readable medium storing a set of instructions, the set of instructions comprising:
one or more instructions that, when executed by one or more processors of a device, cause the device to:
receive source compound simplified molecular-input line-entry (SMILE) data, target compound SMILE data, and a latent space representing compounds;
identify the source compound SMILE data;
project the source compound SMILE data and the target compound SMILE data into the latent space to generate a source compound tensor and a target compound tensor, respectively;
process the source compound tensor, with one or more pretrained models, to determine a reward for the source compound tensor;
determine, based on the reward, a direction and a magnitude to move in the latent space from the source compound tensor;
move the direction and the magnitude in the latent space to a new compound tensor;
determine whether the new compound tensor matches the target compound tensor; and
return a policy based on the new compound tensor matching the target compound tensor.

16. The non-transitory computer-readable medium of claim 15, wherein the one or more instructions, that cause the device to process the source compound tensor, with the one or more pretrained models, to determine the reward for the source compound tensor, cause the device to:
calculate one or more estimates associated with one or more properties of the source compound tensor;
determine one or more heuristics associated with the source compound tensor;
calculate a distance between the source compound tensor and the target compound tensor; and
combine the one or more estimates, the one or more heuristics, and the distance together to determine the reward for the source compound tensor.

17. The non-transitory computer-readable medium of claim 15, wherein the one or more instructions, that cause the device to determine the direction and the magnitude to move in the latent space from the source compound tensor, cause the device to:
determine the direction based on a dimension of the latent space; and
determine the magnitude based on dimensions of the latent space, a quantity of allowed moves, and positive or negative directions.

18. The non-transitory computer-readable medium of claim 15, wherein the one or more instructions further cause the device to:
process the new compound tensor, with the one or more pretrained models and based on the new compound tensor failing to match the target compound tensor, to determine another reward for the new compound tensor;
determine, based on the other reward, another direction and another magnitude to move in the latent space from the new compound tensor;
move the other direction and the other magnitude in the latent space to another new compound tensor;
determine whether the other new compound tensor matches the target compound tensor; and
return another policy based on the other new compound tensor matching the target compound tensor.

19. The non-transitory computer-readable medium of claim 15, wherein the one or more instructions further cause the device to:
identify one or more new compounds based on the policy; and
provide data identifying the one or more new compounds for display.

20. The non-transitory computer-readable medium of claim 15, wherein the one or more instructions further cause the device to:
identify one or more new compound tensors based on the policy;
generate one or more new compound SMILE data based on the one or more new compound tensors; and
provide the one or more new compound SMILE data for display.
